(19) United States
(12) Patent Application Publication
(10) Pub. No.: US 2022/0189578 A1
(43) Pub. Date: Jun. 16, 2022

(54) INTERPRETABLE MOLECULAR GENERATIVE MODELS

(71) Applicant: International Business Machines Corporation, Armonk, NY (US)

(72) Inventor: Seiji Takeda, Minato-ku (JP)

(21) Appl. No.: 17/121,212

(22) Filed: Dec. 14, 2020

(51) Int. Cl. G16B 15/00; G16B 40/00; G06N 3/08

(52) U.S. Cl. CPC G16B 15/00; G06N 3/08; G16B 40/00

(57) ABSTRACT

An approach to training a molecule generative model with interpretable a latent space to identify substructures for a generated molecule generative from the latent space generated from an input molecule with a target property may be provided. A molecule generative model may be trained with a dataset of molecular structures with associated properties and known substructures. The model may generate a latent space in which a substructure predictor model may further be trained to predict the number of substructures of a molecule with target properties from an input molecule with the target properties and identified substructures.

### BACKGROUND OF THE INVENTION

The present invention relates generally to the field of molecular structure generative models, and more specifically to substructure identification in molecular structures generated by a molecule generative model.

Designing new compounds can be a labor intensive and expensive process. In many cases determining if a new compound can be utilized for an intended purpose is determined by trial and error. Progress of chemist or chemical engineer performing wet experimentation is limited and it is unrealistic to test out every possible compound. Quicker development of compounds with known properties is desirable in numerous industries including automotive, pharmaceutical, aviation, semiconductor, and agriculture. Currently, there are numerous libraries with molecular structures possessing physical and chemical properties available to researches. Generative models can assist researchers in narrowing the search for molecular structures with desired properties. Machine learning techniques have allowed for increasingly large amounts of data to be analyzed and processed, including databases of molecular structures.

### SUMMARY

Embodiments of the present disclosure include a computer-implemented method, computer program product, and a system for training a molecular generative model. The embodiments include training a machine learning model with a dataset of molecular structures to generate an output molecular structure with a target property based on an input molecular structure with the target property. Further, embodiments include generating a latent space from the dataset of molecular structures. Additionally, embodiments include training a substructure prediction model to predict one or more substructures of the output molecular structure with the target property based on the generated latent space of the input molecular structure.

The disclosure also provides embodiments for generating a candidate molecule with target properties and the number of predicted substructures of generated molecule associated with the target property. Embodiments include generating a latent space for an input molecule with a molecule generative model. Further, embodiments include predicting one or more substructures of an output molecule with the one or more target properties with a substructure prediction model trained to predict one or more substructures from the latent space generated by the molecule generative model, based on the input molecule.

The above summary is not intended to describe each illustrated embodiment of every implementation of the present disclosure.

### BRIEF DESCRIPTION OF THE DRAWINGS

FIG. 1 is a functional block diagram generally an interpretable molecular structure generative model environment, in accordance with an embodiment of the present invention.

FIG. 2 is a functional block diagram depicting a molecule generative engine, in accordance with an embodiment of the present invention.

FIG. 3 is a flowchart depicting a method for training an interpretable molecular generative model and a substructure prediction model to predict substructures for a molecular structure generated by the molecule generative model, in accordance with an embodiment of the present invention.

FIG. 4 is a flowchart depicting a method for predicting the number of a substructure within a generated molecular structure.

FIG. 5 is a functional block diagram of an exemplary computing system within an interpretable molecular structure generative model environment, in accordance with an embodiment of the present invention.

FIG. 6 is a diagram depicting a cloud computing environment, in accordance with an embodiment of the present invention.

FIG. 7 is a functional block diagram depicting abstraction model layers, in accordance with an embodiment of the present invention.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

The embodiments depicted allow for an interpretable molecular structure generative model, more specifically training a machine learning model to generate a candidate molecular structure with a target property based on an input molecule and predict a property associated with the candidate molecular structure. Further, embodiments provide an approach for jointly training a machine learning model to interpret the latent space of the molecule generative model to predict the number of a substructure associated with the target property in the candidate molecular structure.

Constant improvements in materials development have greatly benefitted humanity. Enormous libraries of molecules and the associated molecular structures and properties for the molecules have been assembled. These libraries have allowed researchers to review and attempt creating new chemical compounds. However, developing these new compounds is time consuming, costly, and at times dangerous. Recent advances in machine learning methods have allowed for the analysis of huge amounts of data. This includes the realm of materials and compound development. One such machine learning method is a neural network. Using a neural network or other machine learning methods to develop and identify new molecules with desired properties is an efficient way to accomplish this task. Using a deep neural network (“DNN”) as an inverse molecular design system can be an effective approach to generate candidate molecules from input molecules with desired traits. However, there is not a way for researchers to understand the hidden layers and latent space of a deep neural network. This is due to the information within a latent space being mixed or entangled within the layers. An approach to predict the number of a substructure for a candidate molecular structure generated from the latent space would allow researchers to improve the process of identifying new candidate molecular structures.

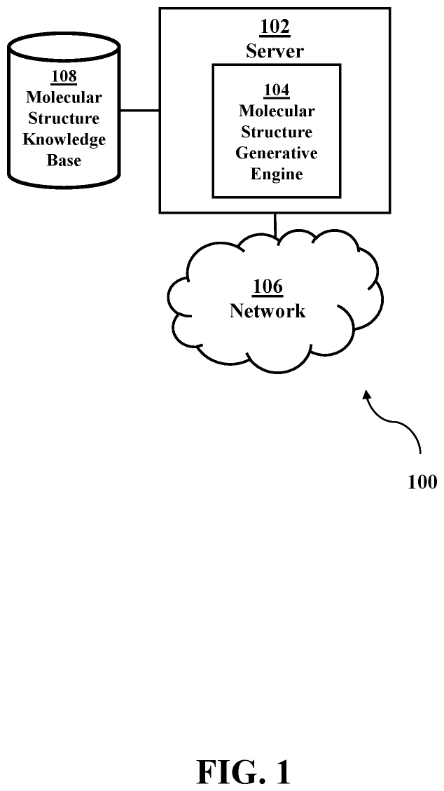

FIG. 1 is a functional block diagram depicting an interpretable molecular structure generative model environment 100. Interpretable molecular structure generative model environment 100 comprises molecular structure generative engine 104 operational on server 102 and molecular structure knowledge base 108 stored on server 102 and network 106.

Server 102 can be a standalone computing device, a management server, a web server, a mobile computing device, or any other electronic device or computing system capable of receiving, sending, and processing data. In other embodiments, server 102 can represent a server computing system utilizing multiple computers as a server system. In another embodiment, server 102 can be a laptop computer, a tablet computer, a netbook computer, a personal computer, a desktop computer, or any programmable electronic device capable of communicating with other computing devices (not shown) within interpretable molecular structure generative model environment 100 via network 106.

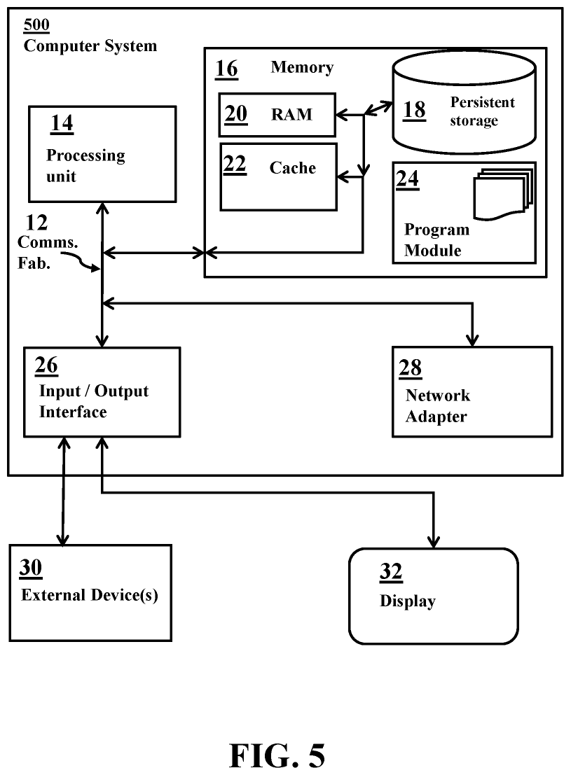

In another embodiment, server 102 represents a computing system utilizing clustered computers and components (e.g., database server computers, application server computers, etc.) that can act as a single pool of seamless resources when accessed within interpretable molecule structure generative model environment 100. Server 102 can include internal and external hardware components, as depicted and described in further detail with respect to FIG. 5.

Molecular structure generative engine 104 can be a computer module capable receiving one or more molecular structure datasets. Further, molecular structure generative engine 104 can be configured to generate a candidate molecular structure with one or more target properties and predict the number of one or more substructures within the candidate molecular structure. (described further below). In some embodiments, molecular structure generative engine 104 can be a DNN with multiple layers or multiple neural networks wherein a subsequent neural network receives the output of a previous neural network to generate a candidate molecular structure. It should be noted, while in FIG. 1 molecular structure generative engine 104 is shown operational on server 102, it may be operational on multiple computing devices (not shown) within interpretable molecular structure generative model environment 100, via network 106. Additionally, in some embodiments, a user may access molecular structure generative engine 104 from a client computer in communication with server 102 (not shown), via a user interface configured to receive an input molecular structure with a target property.

Molecular structure generative engine 104 may comprise a variational auto encoder. A variational auto encoder is a machine learning model composed of an encoder and a decoder. The encoder can be a neural network configured to receive an input of feature vectors of a molecular structure. In some embodiments, a molecular structure in simple molecular input line entry system (“SMILES”) format may be entered into the variational autoencoder.

Molecular structure knowledge base 108 can be a database capable of storing data associated with molecules, including molecular structure. Molecular structure knowledge base can be a preexisting database (e.g., ZINC 15, QM9, etc.). The data associated with the molecules may include physical or chemical properties of molecules in their various structures (e.g., molecular weight, water-octanol partition coefficient (“log P”), rotatable bonds, energy gap, quantitative estimation of drug-likeness (“QED”), synthetic accessibility score (“SAS”), electronic spatial properties (“R²”), etc.). The molecular structure data may include which atoms make up a molecule and the spatial arrangement of the molecule. Further, data associated with molecules can include substructures (e.g., hydroxyl group, benzyl group, carboxyl group, methyl group, ketone group, phenol group, amino group, ect.) and the number of substructure groups within the molecule. Additional data may include the syntax which the molecular structure is expressed (e.g., SMILES, InChI, etc.). It should be noted, FIG. 1 shows Molecular structure knowledge base 108 located on server 102, in some embodiments Molecular structure knowledge base 108 may be located on one or more computing devices or within a cloud computing system.

Network 106 can be, for example, a local area network (LAN), a wide area network (WAN) such as the Internet, or a combination of the two, and can include wired, wireless, or fiber optic connections. In general, network 106 can be any combination of connections and protocols that will support communications between server 102 and other computing devices (not shown).

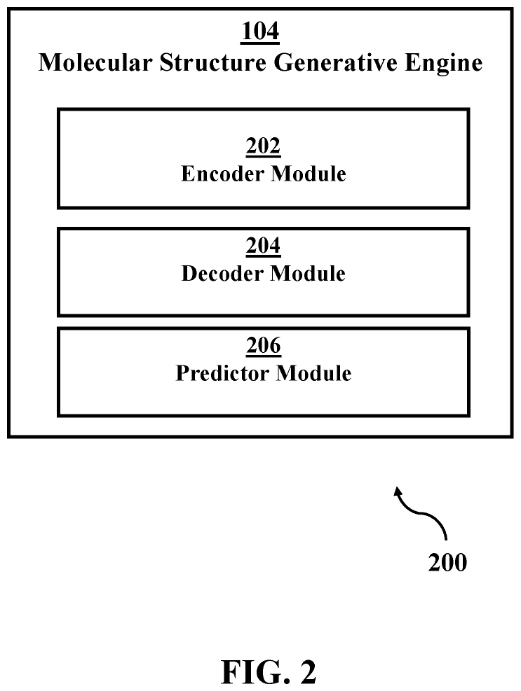

FIG. 2 is functional block diagram 200 of molecular structure generative engine 104. Operational on molecular structure generative engine can be encoder module 202, decoder module 204, and predictor module 206. In some embodiments, molecular structure generative engine 104 may be a variational auto encoder. A variational auto encoder is comprised of an encoding portion and a decoding portion. Further, embodiments of molecular structure generative engine 104 can be comprised of predictor models configured to analyze the latent space generated by an encoder and predict information (e.g., properties and number of substructures).

Encoder module 202 is a computer module that can be configured to receive an input molecular structure with a desired property and generate a latent space based on the molecular structure. A latent space is the compressed data within a machine learning model. It should be noted, in this specification latent space is also referred to as z-space or hidden layer and the terms can be used interchangeably. For example, in a neural network the latent space would be a final output layer of vector representations, in which the statistically significant information is retained. In some embodiments, encoder module 202 can be a module composed of multiple neural networks. Further, a discrete representation of a molecular structure may be received at encoder module 202. For example, a user may enter a molecular structure into encoder module 202. The encoder module 202 may be configured to receive molecular structures in SMILES syntax. In SMILES syntax the molecular structure is represented by alphabetical characters and symbols representing specific bonds. The encoder module 202 can generate a vector representation from the input of the molecular structure in SMILES Syntax. In some embodiments, the encoder module 202 can be a recurrent neural network with a configured to accept SMILES inputs and a filter configured to analyze a preconfigured number of characters in sequence.

An example of an encoder architecture according to an embodiment of the present invention, can be as follows for a SMILES input: an input layer configured for up to 87 nodes wide with 30 potential input characters, followed by three, one-dimensional convolutional layers with filter sizes of 27, 18, and 9 respectively. The next layer is composed of two parallel networks fully connected to the output layer of the final convolutional layer. One network is 128 nodes wide configured to calculate the mean of the vectors from the final convolutional layer (e.g., z_m). The other parallel layer is 128 nodes wide configured to calculate a standard deviation (e.g., z_σ) from the output of the final convolutional layer. The final output layer is a single layer 128 nodes that are fully connected to the two outputs and includes sampling with gaussian noise.

Decoder module 204 is a computer module that can be configured to receive output vector representations of a molecular structure from encoder module 202 and convert the vector representation into a candidate molecular structure. Because the latent space generated by encoder module 202 contains all statistically significant data of the input molecule, decoder module 204 can be configured to output a molecule that is different from the input molecule but possesses all of the same statistically significant data. In some embodiments, decoder module 204 can be a machine learning module configured to convert the latent space of an encoder into a molecule. Further, the machine learning module can be a neural network model, optimized to generate a new candidate model with a target property. In some embodiments, the neural network can be a deep neural network with multiple layers, in which the layers can be the same or different types of neural networks (e.g., gated recurrent neural network, convolutional network, feedforward network). In an embodiment, decoder module 204 can be configured to output a molecular structure in SMILES syntax. Further, a decoder is not required to output in the same syntax format as an encoder input but can be configured to output in any molecular structure syntax format desired, due to the nature of the data contained in the latent space.

An example of a decoder architecture, according to an embodiment of the present invention, can be as follows for a SMILES output: a latent space of vector representations for a molecular structure can be received at a gated recurrent unit with hidden dimension of 512 nodes, followed by a second gated recurrent unit with hidden dimension of 256, followed by a third gated recurrent unit with hidden dimension of 128. The third gated recurrent unit is followed by a fully connected layer of 87 nodes with 30 potential output variables.

Predictor module 206 is a computer module that can be configured to calculate the properties of a candidate molecular structure generated by a decoder, based on the latent space. Predictor module 206 can also be configured to predict the number of a specific substructure within a generated candidate molecular structure based on the latent space generated by an encoder module 202. It should be noted predictor module 206 can perform the operation of predicting one or more properties for a generated candidate molecule in parallel with predicting the substructure number and or/type based on the latent space. In some embodiments, predictor module 206 can predict a property prior to predicting the substructure predictions. In some embodiments, property predictor functionality of predictor module 206 can be a machine learning model. The property predictor can be configured to predict whether a candidate molecular structure has a specific property (e.g., log P, rotatable bonds, energy gap, quantitative estimation of drug-likeness (“QED”), SAS, R², etc.) Additionally, the machine learning model can be a neural network with multiple architectures using multiple nodes and filters. Further, in some embodiments, a neural network can be trained for each substructure based on a generated latent space.

An example of a neural network for a property predictor can be as follows. An input layer can receive the latent space, the layer can be fully connected to the latent space and have 128 nodes. A fully connected layer of 64 nodes can follow the input layer. An output layer of one node can be connected to the previous layer of 64 nodes. This layer can be a probability predictor of the candidate molecular structure for the desired property.

An example of a neural network for a substructure property predictor can be as follows: an input layer can receive the vector representations from the latent space of an encoder, it can be a fully connected 128 nodes layer. A fully connected layer of 64 nodes can follow the input layer. An output layer of one node can be connected to the previous layer of 64 nodes. This layer can be a probability predictor of the candidate molecular structure for the number of substructures in which the substructure has been trained to identify from the latent space.

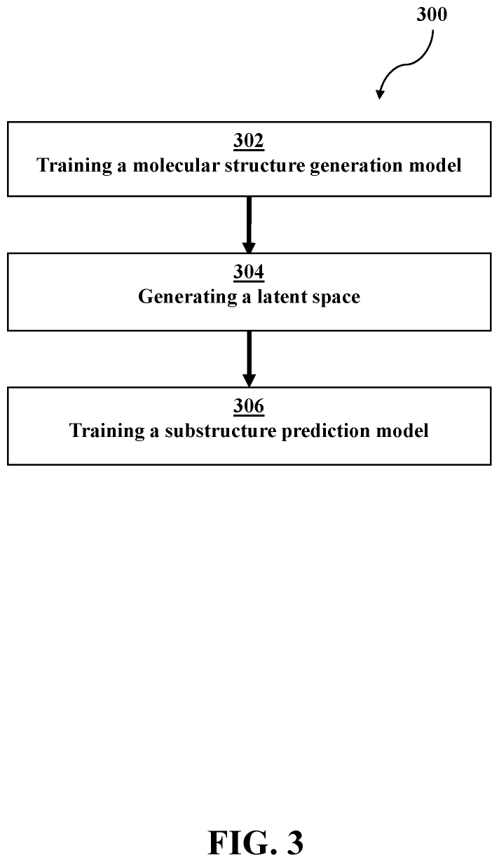

FIG. 3 is a flowchart depicting a method 300 for training an interpretable molecule generative model. At step 302, train a molecule generative model. In some embodiments, a machine learning model operational on molecular structure generative engine 104 can receive a dataset of molecular structures from molecular structure database. The molecule generative model can be optimized for molecule generative based on a gradient descent, where the sample molecular structure has a known property, and the generated molecule has a similar molecular structure and the same or similar property.

At step 304, a latent space can be generated via molecular structure generative engine 104 from the training data received. In some embodiments, encoder module 202 can be a variational autoencoder which can be optimized from the known molecular structures within the training dataset. The latent space can be a continuous vector representation of each sample within the dataset.

At step 306, jointly-train a model to predict the number of substructures from the latent space. In some embodiments, predictor module 206 is trained to identify the number of a specific type of substructure from the latent space. For example, samples within the dataset can have a known number of specific substructures within the molecular structure. The latent space of molecular structures with a hydroxyl group can be used to train the predictor model. Further, in some embodiments, sets of local latent units can be assigned to correspond to a specific substructure. The sets can be selected sparsely within the latent space.

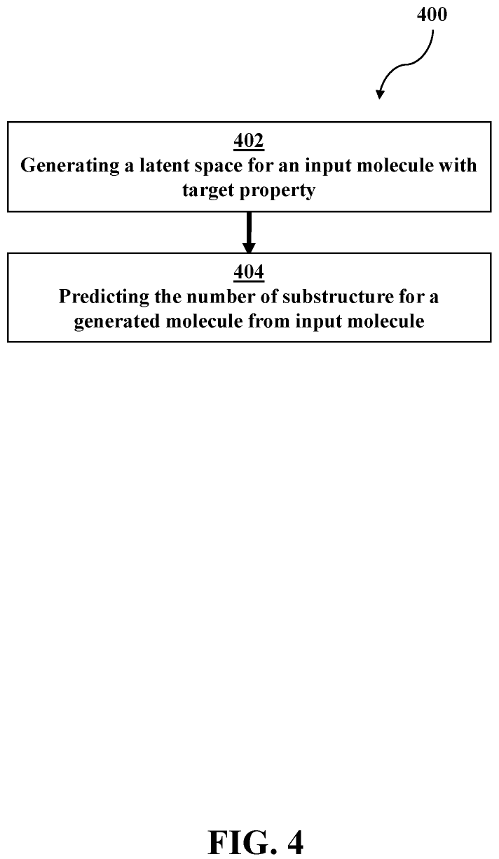

FIG. 4 is a flowchart depicting a method 400 for generating a candidate molecule with predicted substructures and target properties from an input molecule with a target molecule with an interpretable molecule generative model. At step 402, generate a latent space for an input molecule. In some embodiments, a user can input a molecular structure in discrete representation form into molecular structure generative engine 104. Further, encoder module 202 can generate a latent space of continuous representation vectors for the input molecule. Decoder module 204 can convert the latent space into a candidate molecule with the target property. In some embodiments, decoder module can be a neural network with multiple layers configured to convert the continuous vector representations generated by a neural network in the encoder module 202.

At step 404, predict the number of a substructure for a molecular structure generated by predictor module 206. In some embodiments, a candidate molecular structure with target properties is generated from the latent space generated by encoder module 202. Predictor module 206 can receive the latent space for an input molecular structure and predict the number of a specific type of atomic substructure for the molecular structure generated from the same latent space. For example, a molecular structure with a large log P may have an indicated hydroxyl substructure. Predictor module may predict the substructure has three such hydroxyl substructures.

FIG. 5 depicts computer system 500, an example computer system representative of server 102 and molecular structure knowledge base 108 or any other computing device within an embodiment of the invention. Computer system 500 includes communications fabric 12, which provides communications between computer processor(s) 14, memory 16, persistent storage 18, network adaptor 28, and input/output (I/O) interface(s) 26. Communications fabric 12 can be implemented with any architecture designed for passing data and/or control information between processors (such as microprocessors, communications and network processors, etc.), system memory, peripheral devices, and any other hardware components within a system. For example, communications fabric 12 can be implemented with one or more buses.

Computer system 500 includes processors 14, cache 22, memory 16, network adaptor 28, input/output (I/O) interface(s) 26 and communications fabric 12. Communications fabric 12 provides communications between cache 22, memory 16, persistent storage 18, network adaptor 28, and input/output (I/O) interface(s) 26. Communications fabric 12 can be implemented with any architecture designed for passing data and/or control information between processors (such as microprocessors, communications and network processors, etc.), system memory, peripheral devices, and any other hardware components within a system. For example, communications fabric 12 can be implemented with one or more buses or a crossbar switch.

Memory 16 and persistent storage 18 are computer readable storage media. In this embodiment, memory 16 includes persistent storage 18, random access memory (RAM) 20, cache 22 and program module 24. In general, memory 16 can include any suitable volatile or non-volatile computer readable storage media. Cache 22 is a fast memory that enhances the performance of processors 14 by holding recently accessed data, and data near recently accessed data, from memory 16. As will be further depicted and described below, memory 16 may include at least one of program module 24 that is configured to carry out the functions of embodiments of the invention.

The program/utility, having at least one program module 24, may be stored in memory 16 by way of example, and not limiting, as well as an operating system, one or more application programs, other program modules, and program data. Each of the operating systems, one or more application programs, other program modules, and program data or some combination thereof, may include an implementation of a networking environment. Program module 24 generally carries out the functions and/or methodologies of embodiments of the invention, as described herein.

Program instructions and data used to practice embodiments of the present invention may be stored in persistent storage 18 and in memory 16 for execution by one or more of the respective processors 14 via cache 22. In an embodiment, persistent storage 18 includes a magnetic hard disk drive. Alternatively, or in addition to a magnetic hard disk drive, persistent storage 18 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 18 may also be removable. For example, a removable hard drive may be used for persistent storage 18. Other examples include optical and magnetic disks, thumb drives, and smart cards that are inserted into a drive for transfer onto another computer readable storage medium that is also part of persistent storage 18.

Network adaptor 28, in these examples, provides for communications with other data processing systems or devices. In these examples, network adaptor 28 includes one or more network interface cards. Network adaptor 28 may provide communications through the use of either or both physical and wireless communications links. Program instructions and data used to practice embodiments of the present invention may be downloaded to persistent storage 18 through network adaptor 28.

I/O interface(s) 26 allows for input and output of data with other devices that may be connected to each computer system. For example, I/O interface 26 may provide a connection to external devices 30 such as a keyboard, keypad, a touch screen, and/or some other suitable input device. External devices 30 can also include portable computer readable storage media such as, for example, thumb drives, portable optical or magnetic disks, and memory cards. Software and data used to practice embodiments of the present invention can be stored on such portable computer readable storage media and can be loaded onto persistent storage 18 via I/O interface(s) 26. I/O interface(s) 26 also connect to display 32.

Display 32 provides a mechanism to display data to a user and may be, for example, a computer monitor or virtual graphical user interface.

The components described herein are identified based upon the application for which they are implemented in a specific embodiment of the invention. However, it should be appreciated that any particular component nomenclature herein is used merely for convenience, and thus the invention should not be limited to use solely in any specific application identified and/or implied by such nomenclature.

The present invention may be a system, a method and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems) and computer program products according to embodiments of the invention. It is understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality and operation of possible implementations of systems, methods, and computer program products according to various embodiments of the present invention. In this regard, each block in the flowchart or block diagrams may represent a module, segment or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

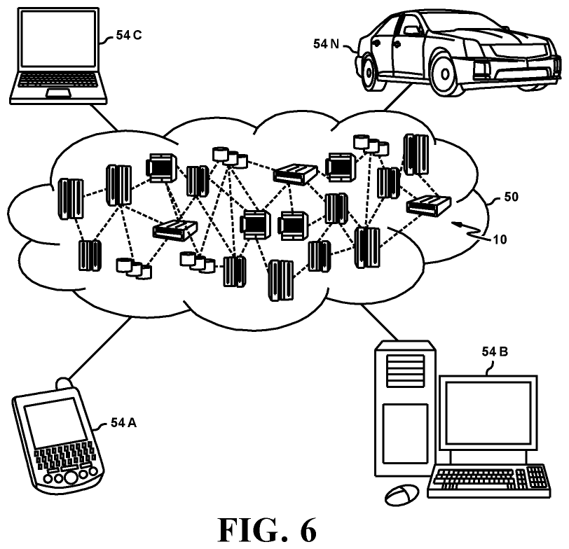

FIG. 6 is a block diagram depicting a cloud computing environment 50 in accordance with at least one embodiment of the present invention. Cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

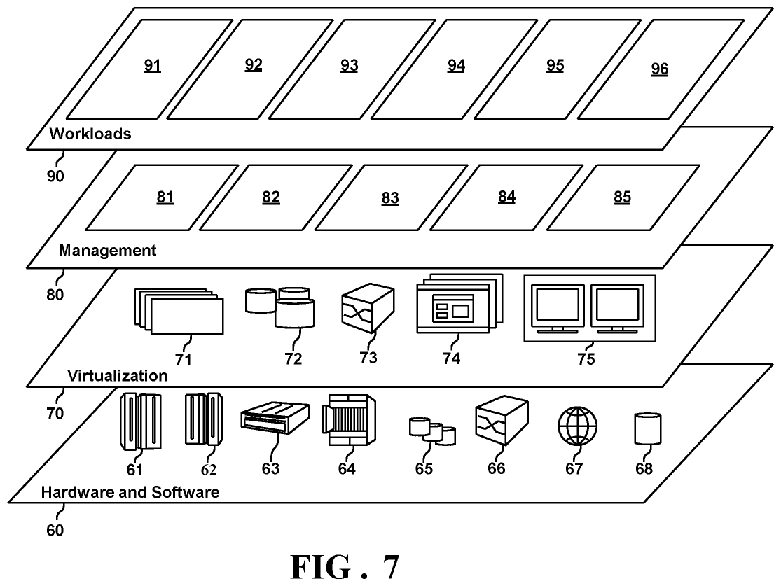

FIG. 7 is a block diagram depicting a set of functional abstraction model layers provided by cloud computing environment 50 depicted in FIG. 6 in accordance with at least one embodiment of the present invention. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and interpretable molecular structure generative 96.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method for training a molecule generative model, the method comprising:
training, by one or more processors, a molecule generative model with a dataset of molecular structures to generate an output molecular structure with a target property based on an input molecular structure with the target property;
generating, by the one or more processors, a latent space from the dataset of molecular structures; and
training, by the one or more processors, a substructure prediction model to predict one or more substructures of the output molecular structure with the target property based on the generated latent space of the input molecular structure.

2. The computer-implemented method of claim 1, wherein the molecule generative model is a variational autoencoder.

3. The computer-implemented method of claim 1, wherein the syntax of the molecular structure for the input molecular structure, output molecular structure, and dataset of molecular structures within the molecule generative model is simple molecular input line entry.

4. The computer-implemented method of claim 1, wherein the dataset of molecular structures is comprised of a plurality of molecular structures, wherein each molecular structure has property data and substructure data.

5. The computer-implemented method of claim 1, wherein training the substructure prediction model to predict one or more substructures further comprises:
assigning, by the one or more processors, a set of local latent units within the latent space sparsely with a plurality of substructures associated with molecular structures from the molecular structure dataset.

6. A system for training a molecule generative model, the system comprising:
one or more computer processors;
one or more computer readable storage media; and
computer program instructions to:
train a molecule generative model with a dataset of molecular structures to generate an output molecular structure with a target property based on an input molecular structure with the target property;
generate a latent space from the dataset of molecular structures; and
train a substructure prediction model to predict one or more substructures of the output molecular structure with the target property based on the generated latent space of the input molecular structure.

7. The system of claim 6, wherein the molecule generative model is a variational autoencoder.

8. The system of claim 6, wherein the syntax of the molecular structure for the input molecular structure, output molecular structure, and dataset of molecular structures within the molecule generative model is simple molecular input line entry.

9. The system of claim 6, wherein the dataset of molecular structures is comprised of a plurality of molecular structures, wherein each molecular structure has property data and substructure data.

10. The system of claim 6, wherein training the substructure prediction model to predict one or more substructures further comprises:
assigning, by the one or more processors, a set of local latent units within the latent space sparsely with a plurality of substructures associated with molecular structures from the molecular structure dataset.

11. A computer program product comprising a computer readable storage medium having program instructions embodied therewith, the program instructions executable by a processor to cause the processors to perform a function, the function comprising:
train a molecule generative model with a dataset of molecular structures to generate an output molecular structure with a target property based on an input molecular structure with the target property;
generate a latent space from the dataset of molecular structures; and
train a substructure prediction model to predict one or more substructures of the output molecular structure with the target property based on the generated latent space of the input molecular structure.

12. The computer program product of claim 11, wherein the molecule generative model is a variational autoencoder.

13. The computer program product of claim 11, wherein the syntax of the molecular structure for the input molecular structure, output molecular structure, and dataset of molecular structures within the molecule generative model is simple molecular input line entry.

14. The computer program product of claim 11, wherein the dataset of molecular structures is comprised of a plurality of molecular structures, wherein each molecular structure has property data and substructure data.

15. The computer program product of claim 11, wherein training the substructure prediction model to predict one or more substructures further comprises:
assigning, by the one or more processors, a set of local latent units within the latent space sparsely with a plurality of substructures associated with molecular structures from the molecular structure dataset.

16. A computer-implemented method for generating a molecule with target properties and number of substructures of generated molecule associated with the target property, the method comprising:
generating, by the one or more processors, a latent space for an input molecule with a molecule generative model; and
predicting, by the one or more processors, one or more substructures of an output molecule with the one or more target properties with a substructure prediction model trained to predict one or more substructures from the latent space generated by the molecule generative model, based on the input molecule.

17. The computer-implemented method of claim 16, wherein the molecule generative model is a neural network.

18. The computer-implemented method of claim 17, wherein the neural network is an autoencoder.

19. The computer implemented method of claim 16, wherein the substructure prediction model is a decoding neural network.

20. The computer-implemented method of claim 16, further comprising:
generating, by one or more processors, an output molecule with the at least one target properties with the molecule generative model, with the molecule generative model.

21. A system for generating a molecule with target properties and number of substructures of generated molecule associated with the target property, the system comprising:
one or more computer processors;
one or more computer readable storage media; and
computer program instructions to:
generate a latent space from an input molecule with a molecule generative model; and
predicting, by the one or more processors, one or more substructures of an output molecule with the one or more target properties with a substructure prediction model trained to predict one or more substructures from the latent space generated by the molecule generative model, based on the input molecule.

22. The system of claim 21, wherein the molecule generative model is a neural network.

23. The system of claim 22, wherein the neural network is an autoencoder.

24. The system of claim 21, wherein the substructure prediction model is a decoding neural network.

25. The system of claim 21, further comprising instructions to:
generating, by one or more processors, an output molecule with the at least one target properties with the molecule generative model, with the molecule generative model.
